(19) United States
(12) Patent Application Publication
(10) Pub. No.: US 2016/0235500 A1
(43) Pub. Date: Aug. 18, 2016

(54) TOOL FOR THE ACTIVATION OF PALATAL EXPANDERS

(71) Applicant: LEONE S.P.A., Sesto Fiorentino (FI) (IT)

(72) Inventors: Maurizio DOLFI, Firenze (IT); Gabriele SCOMMEGNA, Tavarnuzze Impruneta, Firenze (IT)

(73) Assignee: Leone S.P.A.

(21) Appl. No.: 15/026,335

(22) PCT Filed: Sep. 5, 2014
(86) PCT No.: PCT/IT2014/000236

§ 371 Date: Mar. 31, 2016

(30) Foreign Application Priority Data
Oct. 1, 2013 (IT) FI2013A000227

(51) Int. Cl. A61B 90/00; A61C 7/10; A61C 7/02

(52) U.S. Cl. CPC A61B 90/08; A61C 7/02; A61C 7/10; A61B 2090/0803

(57) ABSTRACT

Tool for actuating palatal expanders, comprising a body (1) with a grip portion (2) and, on the opposite side, a tip (3). The body (1) has, on at least one surface (10) thereof, display means adapted for displaying the number of uses.

The present invention relates to a tool, in jargon called “key”, for the activation of palatal expanders. It is known that the palatal expanders are used in the treatment of deficiency of the maxillary arch that require the disjunction of the palatal suture. Such expanders are generally constituted by two bodies slidably mounted on parallel guide rods and constrained, by means of shaped arms, to a predetermined number of teeth of the dental arch to be treated. Said bodies are also threaded to receive, each, a stem of a screw having a double threadening through which the same bodies can be moved away from each other thereby causing the effect of expansion desired. Said screw has a maneuvering or operating head resulting in a central position between said two bodies. The operating head of the screw has a plurality of holes destined to be engaged by a tool, commonly called “key”, allowing the screw to be rotated at predetermined time intervals according to a therapeutic program indicated by the dentist. Usually, the therapeutic program provides for the rotation of the screw of a quarter of a turn at regular time intervals, for example a quarter of a turn once a day for ten days, depending on the type and severity of the deficiency to be corrected. The periodic operation of the screw is generally entrusted to the relatives of the patient. A problem encountered in the use of the screw-operated expanders described above lies in that, especially in the case of treatments particularly prolonged in time, the user does not keep properly track of the number of turns to which the screw has been subjected, and then of the degree of expansion reached, which can be harmful for the patient and may compromise the effectiveness of the treatment.

The main purpose of the present invention is to eliminate, or at least greatly reduce, the drawbacks mentioned above.

This result is achieved, according to the present invention, by providing a device having the characteristics indicated in claim 1. Other features of the present invention are the subject of the dependent claims.

Thanks to the present invention, it is possible to easily keep track of the number of turns or fraction of turns of the screw, without making use of external means, greatly reducing the risk of compromising the efficacy of the treatment. Furthermore, a tool in accordance with the present invention is simple to be made and is inexpensive in relation to the advantages offered.

These and other advantages and features of the present invention will be best understood by anyone skilled in the art thanks to the following description and the accompanying drawings, given by way of example but not to be considered in a limiting sense, wherein:

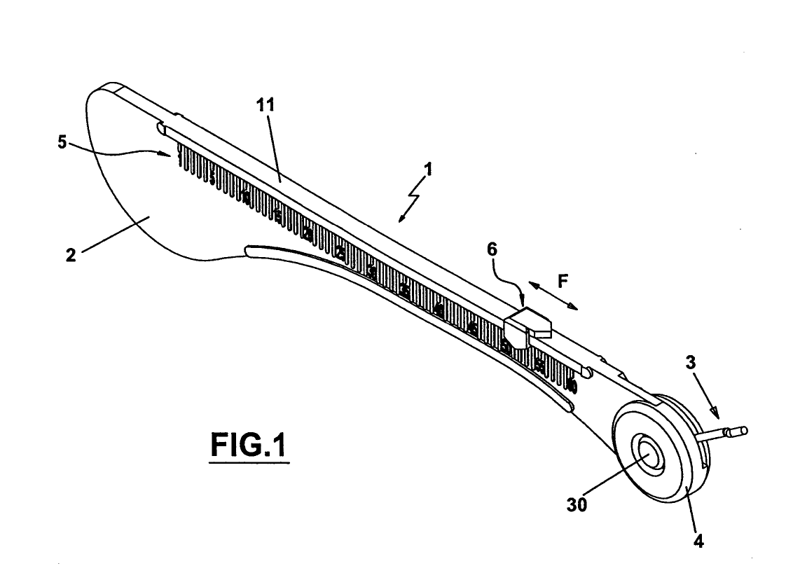

FIG. 1 is a perspective view of a tool in accordance with the present invention;

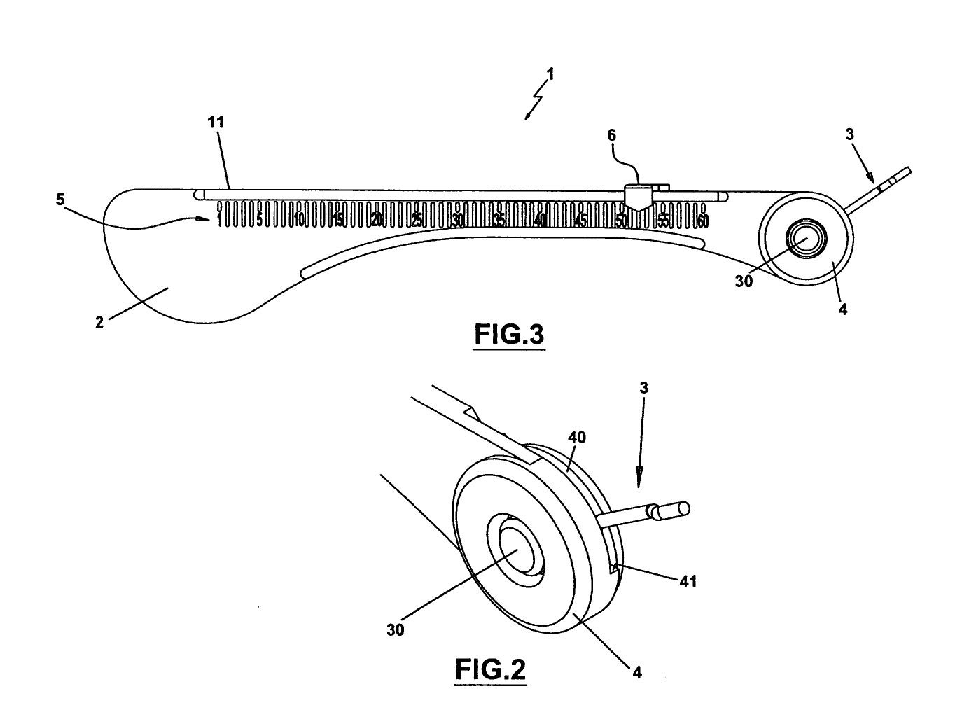

FIG. 2 is an enlarged detail of the tool shown in FIG. 1;

FIG. 3 is a side view of the tool shown in FIG. 1;

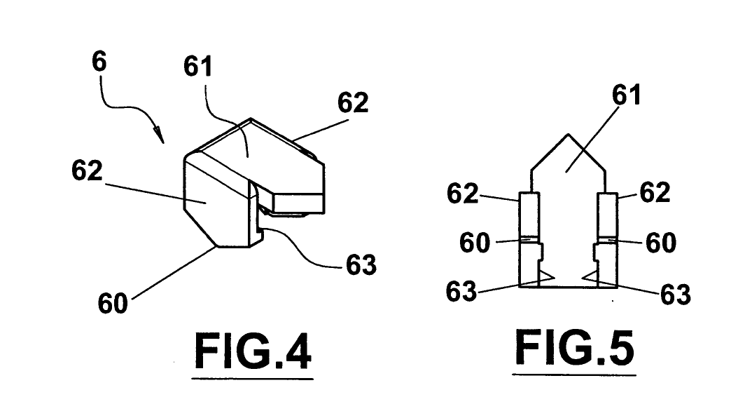

FIG. 4 is a perspective view of the slider (6);

FIG. 5 is a bottom view of the slider shown in FIG. 4;

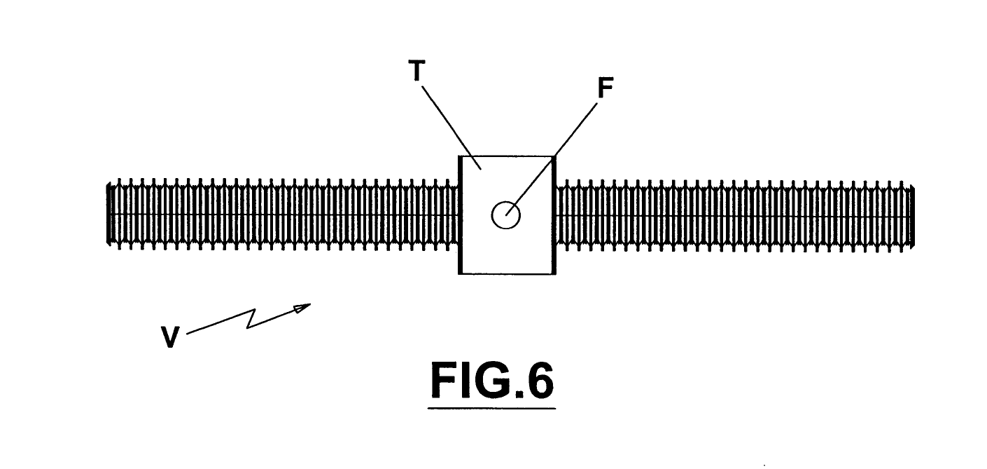

FIG. 6 schematically shows a screw for palatal expanders of the known type;

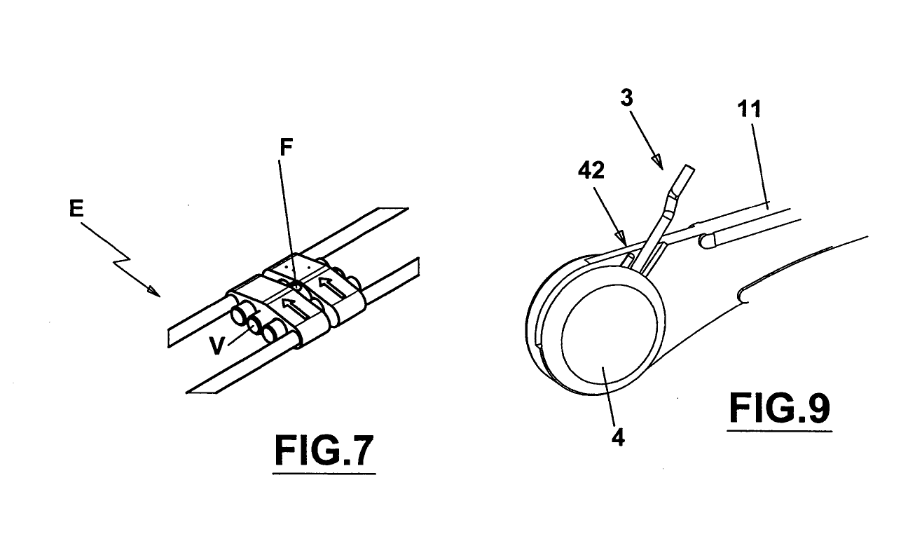

FIG. 7 schematically shows a screw-operated palatal expander of the known type;

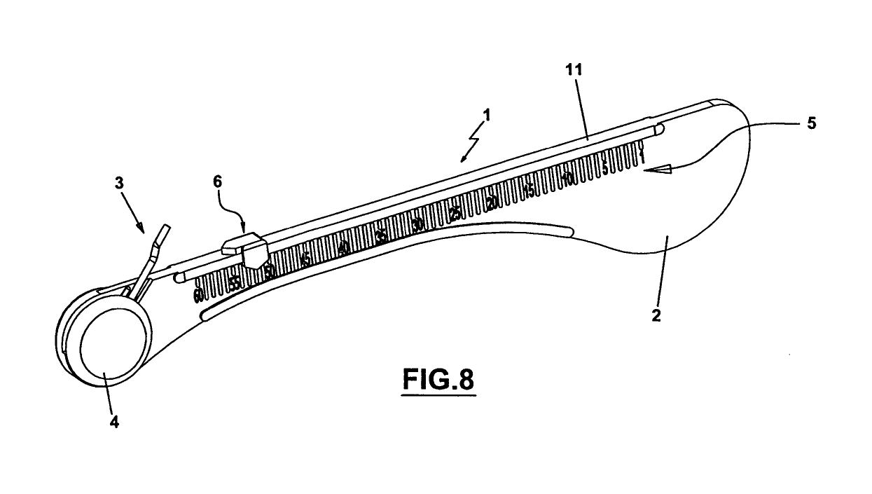

FIG. 8 is another perspective view of the tool shown in FIG. 1;

FIG. 9 shows an enlarged detail of FIG. 8.

Reduced to its basic structure and with reference to the attached drawings, a tool for the activation of a screw-operated palatal expander in accordance with the present invention comprises a body (1) having predominant longitudinal development, i.e. having a length that is greater than the height and thickness, which has a grip portion (2) and, on the opposite side, a tip (3). In this example, the tip (3) is applied, by means of a pin (30) with axis orthogonal to the two sides (10) of the body (1), in a capsule (4) which has a slot (40) within which the same tip (3) can rotate around the axis of the pin (30). The said slot (40) has an abutment point (41) which limits the angular movement of the tip (3) such that the tip (3) can rotate up to interfering with the abutment point. This basic structure of the tool is known per se. Advantageously, in accordance with the present invention, the body (1) has, along at least one of its sides (10), a series of marks (5) and, above said marks (5), a slider (6) that is provided with an appendix (60) facing the same marks (5). The slider (6) is slidingly applied on an upper edge (11) of the body (1) above the marks (5) so that, by sliding along said edge (11) of the body (1), as indicated by arrow “F” in FIG. 1, the aforementioned appendix (60) can be positioned in correspondence of a mark (5).

The marks (5) can be shaped in any way. For example, the marks (5) can be numbers and/or notches provided on one side of the body (1) or on both the left and right sides of the latter, aligned along a direction parallel to that of the edge (11).

The slider (6) is slightly forced on the edge (11) of the body (1) to prevent it from sliding spontaneously.

The slider (6), in accordance with the example shown in the drawings, has an upper surface (61) and two side surfaces (62), the latter being orthogonal to the upper surface (61). The inner side of the upper surface (61) is turned towards the upper edge (11) of the body (1). The side surfaces (62) have each an internal tooth (63) which engages the lower side of the upper edge (11) of the body (1) that, for this purpose, protrudes from both sides of the same body (1). Furthermore, the side surfaces (62) have each two sides converging towards the bottom that define the aforementioned appendix (60). The upper surface (61) is advantageously shaped so as to exhibit a pointed front part that indicates the direction along which the slider (6) must be made to slide.

Each time the tool is used (by inserting the tip 3 into a hole F of the operating head T of the screw V of a screw-operated palatal expander E), the slider (6) is manually advanced along the edge (11) of the body (1), the run of the slider corresponding to the distance between two adjacent marks. In this way, the appendix (60) of the slider (6) moves on the marks (5) recording each time the use of the tool, i.e. the activation of the expander. Therefore, the user is facilitated in keeping track of the number of activations run without external tracking as the tracking mechanism is integrated with the tool.

In practice, a tool in accordance with the present invention is provided with means for displaying the number of uses.

According to the example described above, the means for displaying the number of uses comprise the marks (5) and the slider (6) and the latter has an indicator (60) in correspondence of the marks (5).

It goes without saying, however, that the means for displaying the number of uses may be structured differently.

As shown in FIG. 9, the slot (40) is provided with a protuberance (42) that, when the tip (3) passes in front of it during the activation of the expander, produces a “click” sound alerting the user that the tip (3) has completed a run, and then that the screw (V) has been rotated and consequently the expander (E) has been activated.

In practice the details of execution may vary in any equivalent way as for what concerns the individual elements described and illustrated and to their arrangement without departing from the scope of the adopted solution and thus remaining within the limits of the protection conferred by the present patent.

1. Tool for actuating palatal expanders, comprising a body (1) with a grip portion (2) and, on the opposite side, a tip (3), characterized in that the said body (1) has, on at least one surface(10) thereof, display means adapted for displaying the number of uses.

2. Tool according to claim 1 characterized in that said display means comprise marks (5) on at least one side surface (10) of the body (1) and a slider (6) slidably applied on the same body (1), said slider (6) having an indicator (60) that can be moved on said indices (5).

3. Tool according to claim 2 characterized in that the said marks (5) consist of numbers and/or notches.

4. Tool according to claim 2 characterized in that the said slider (6) is applied on an upper edge (11) of the body (1).

5. Tool according to claim 2 characterized in that said slider (6) has an upper surface (61) and two side surfaces (62) orthogonal to the upper surface (61), in that an inner side of the upper surface (61) is turned towards an upper edge (11) of the body (1), in that the said side surfaces (62) have each an internal tooth (63) which engages the lower side of the upper edge (11) of the body (1), and in that the upper edge (11) of the body (1) protrudes from both sides beyond at least one side of the same body (1).

6. A tool according to claim 1, characterized in that the said tip (3) is applied, by means of a pin (30) with axis orthogonal to two lateral surfaces (10) of the body (1), in a capsule (4) having a slot (40) within which the same tip (3) can rotate around the axis of the pin (30).

7. Tool according to claim 6 characterized in that said slot (40) has an abutment point (41) that limits the angular movement of the tip (3).

8. Tool according to claim 6 characterized in that said slot (40) has an appendix (42) which interferes with the tip (3) when the angular run of the latter is completed thus producing the sound of a click.
